(19) United States
(12) Patent Application Publication
(10) Pub. No.: US 2025/0302453 A1
(43) Pub. Date: Oct. 2, 2025

(54) URINARY CATHETER CONNECTOR ASSEMBLY WITH INFECTION SENSING, AN EXTENSION ASSEMBLY HAVING A CONNECTOR ASSEMBLY, AND METHODS OF USE THEREOF

(71) Applicant: Advocath LLC, Ann Arbor, MI (US)

(72) Inventors: Christine McDunnough, Ann Arbor, MI (US); William Vickers, Holly, MI (US)

(21) Appl. No.: 19/091,701

(22) Filed: Mar. 26, 2025

Related U.S. Application Data
(60) Provisional application No. 63/572,214, filed on Mar. 30, 2024.

(51) Int. Cl. A61B 10/00; A61M 27/00; A61M 39/10

(52) U.S. Cl. CPC A61B 10/007; A61M 39/10; A61B 2010/0006; A61M 27/00; A61M 2202/06

(57) ABSTRACT

An extension assembly including a connector assembly and a hose. The connector assembly includes a housing, a test strip and a hose coupling. The test strip is positionable within the housing, with a portion of the hose coupling insertable into the housing so as to sandwich the test strip therebetween, with a portion of the test strip exposed to the bore of the connector assembly.

### CROSS-REFERENCE TO RELATED APPLICATION

This Application claims priority from U.S.S Provisional Patent App. Ser. No. 63/572,214, entitled “Urinary Catheter Connector Assembly With Infection Sensing, And Extension Assembly Having A Connector Assembly, And Methods Of Use Thereof”, filed Mar. 30 2024, the entire disclosure of which is hereby incorporated by reference in its entirety.

This application is related to, but does not claim priority from U.S. patent application Ser. No. 17/108,838, now U.S. Pat. No. 11,752,298, entitled “Self-Intermittent Urinary Catheter Extension with Infection Detection, A Catheter Assembly Having An Extension With Infection Detection And A Catheter Assembly Having Infection Detection” and also related to U.S. patent application Ser. No. 17/337,350, now U.S. Pat. No. 11,752,299 entitled “Self-Intermittent Urinary Catheter Extension with Infection Detection, A Catheter Assembly Having An Extension With Infection Detection And A Catheter Assembly Having Infection Detection”, the entire disclosure of each of which is hereby incorporated by reference in their entirety.

This application is also related to, but does not claim priority from, U.S. patent application Ser. No. 16/405,961 filed May 7, 2019, entitled “Monolithic Self-Intermittent Catherization System with Attached Extension”. The disclosure of this application is hereby incorporated by reference in its entirety.

### BACKGROUND OF THE DISCLOSURE

### 1. Field of the Disclosure

The disclosure relates in general to urinary catheters, and more particularly, to a connector assembly (and an extension with a connector assembly) that includes infection detection. It will be understood that the system of the present disclosure is not limited to any particular type of catheter, such as a self-intermittent catheters, but can be utilized in association with, among other structures, indwelling catheter systems, without limitation.

### 2. Background Art

The urinary tract consists of the kidneys, bladder, ureters, and urethra. Urine in the bladder is normally sterile. This means it does not contain any bacteria or other germs (such as fungi). But bacteria can enter the urinary tract through the urethra. This can cause a urinary tract infection (UTI). A UTI can happen anywhere in your urinary tract, including your bladder, ureters, kidneys, and urethra. Most UTIs are easy to cure with antibiotics. But an untreated infection can become more severe as it spreads toward the kidneys. An infection in the upper urinary tract is much more challenging to treat. Eventually, the infection can spread into your blood, causing sepsis and frequently, death.

Individuals with spinal cord injuries or other maladies that preclude them from sensation in their lower body have Neurogenic bladders, which necessitates them to void their bladder 5 to 7 times a day through use of a self-intermittent urinary catheter. Because of limited sensation, UTIs are not sensed right away and therefore, treatment is late. There is an extremely high incidence of UTIs and subsequent complications in this population due to these issues, frequently resulting in complications, other medical issues, hospital stays, and often-sepsis and death.

### BRIEF DESCRIPTION OF THE DRAWINGS

The disclosure will now be described with reference to the drawings wherein:

FIG. 1 of the drawings is a side elevational view of the extension assembly of the present disclosure, showing, in particular, the connector assembly and the hose;

FIG. 2 of the drawings is a side elevational and a front plan view of a pair of test strips of the connector assembly of the present disclosure;

FIG. 3 of the drawings is a top plan view of a configuration of the housing of the connector assembly having two test strips mounted therein;

FIG. 4 of the drawings is a composite drawing schematically representing the assembly procedure of the connector assembly of the present disclosure, initially showing a pair of test strips, then a housing wherein the test strips are mounted thereon, the attachment of the hose coupling, and finally, the gluing of the hose coupling to the housing to complete the connector assembly;

FIG. 5 of the drawings is perspective view of the housing of the connector assembly of the present disclosure;

FIG. 6 of the drawings is a side elevational view of the extension assembly

of the present disclosure; and

FIG. 7 of the drawings is a front elevational view of the connector assembly of the present disclosure.

### DETAILED DESCRIPTION OF THE DISCLOSURE

While this disclosure is susceptible of embodiment in many different forms, there is shown in the drawings and described herein in detail a specific embodiment(s) with the understanding that the present disclosure is to be considered as an exemplification and is not intended to be limited to the embodiment(s) illustrated.

It will be understood that like or analogous elements and/or components, referred to herein, may be identified throughout the drawings by like reference characters. In addition, it will be understood that the drawings are merely schematic representations of the invention, and some of the components may have been distorted from actual scale for purposes of pictorial clarity.

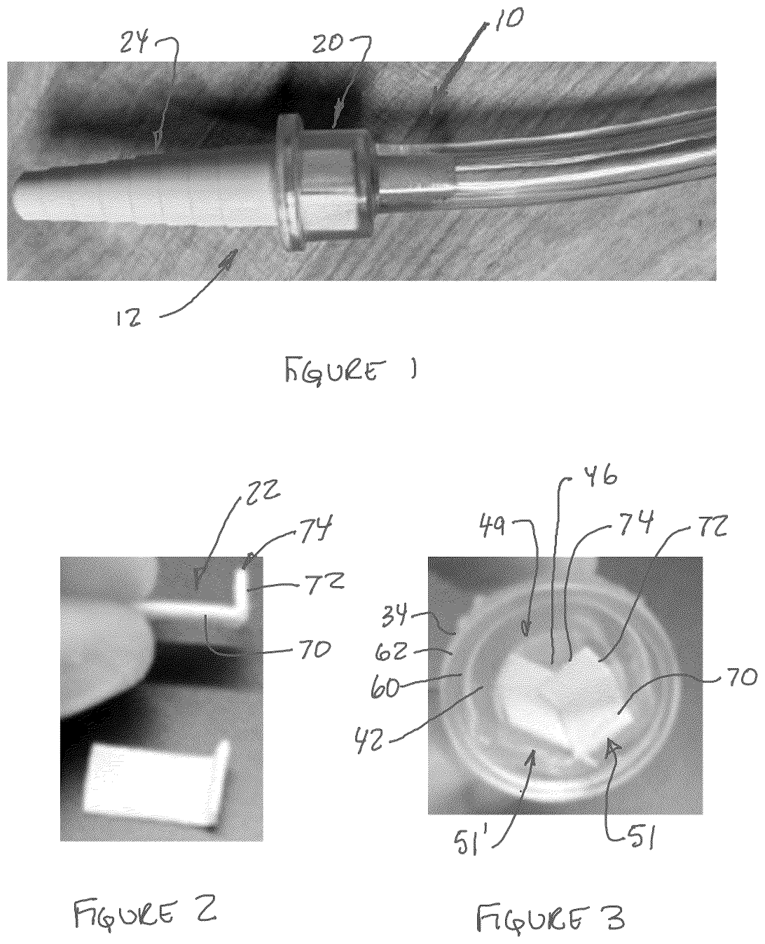

Referring now to the drawings and in particular to FIG. 1, the extension assembly 10 is shown as comprising connector assembly 12 and hose member 14. The connector assembly 12 is configured to provide a coupling to an existing catheter hose, while providing an infection indicator together with the connector assembly, thus providing a convenient device for determining the presence and/or absence of a urinary tract infection.

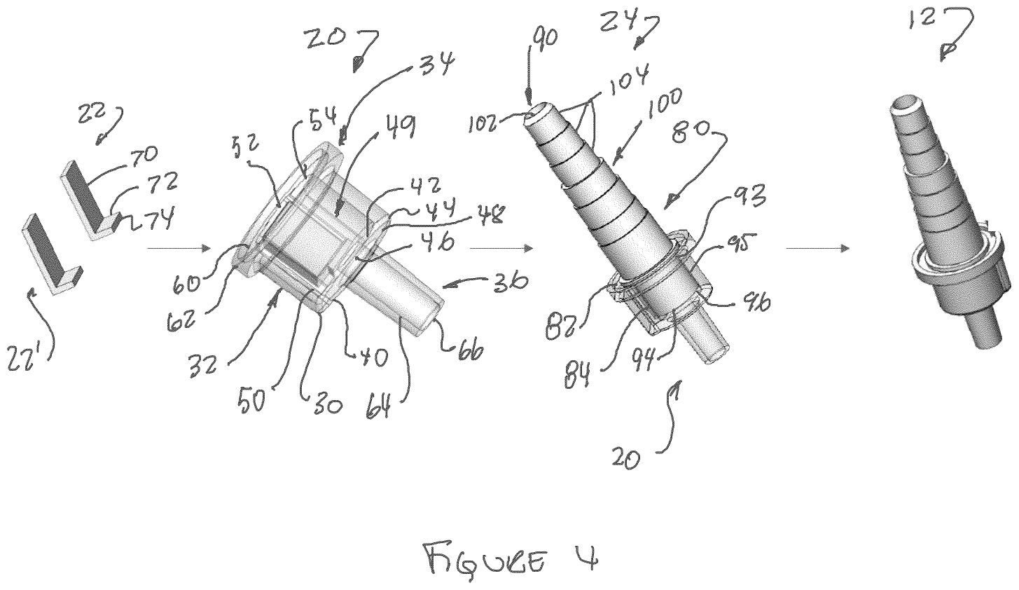

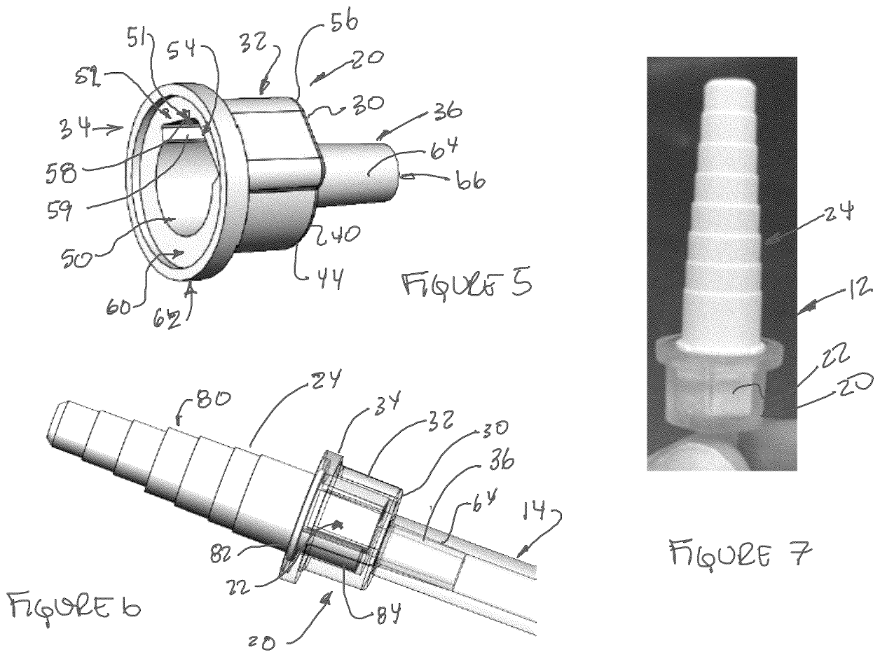

With reference to FIGS. 3 through 5, the connector assembly 12 is shown as comprising housing 20, at least one test strip, such as test strip 22, and hose coupling 24. The at least one test strip (and in the configuration shown, two test strips) is sandwiched between the housing and the hose coupling with access to the bore extending therethrough so as to be in selective contact with fluids (i.e., urine) passing therethrough to indicate the presence or absence of particular constituents that would otherwise indicate the presence (or absence) of an infection.

The housing includes base 30, upstanding wall 32, upper flange 34 and lower hose attachment interface 36. The base 30 includes lower surface 40, upper surface 42, outer perimeter 44. The base 30 further includes an opening 46 which is generally centrally located defining a ring 48 encircling the opening 46. The base 30 is generally circular, with the definition of what is the base wall portion 56, 56′ of the pocket portions 52, 54. The lower hose attachment interface 36 depends from the lower surface 40 of the base 30 defining an outer surface 64 and bore 66.

The upstanding wall 32 extends in a direction opposite the lower hose attachment interface 36, encircling the base 30. The upstanding wall 32 includes first portion 50 (which extends from the generally circular portion), first pocket portion 52 and second pocket portion 54. The first pocket portion 52 and the second pocket portion 54 are positioned, in the configuration shown, next to each other (such that portions of the test strips 22 may be abutting and/or overlapping). In other configurations, the pocket portions may be spaced apart from each other. The first pocket portion and the second pocket portion are generally mirror images of each other, so the first pocket portion will be described with the understanding that the second pocket portion is substantially identical thereto. Thus, similar elements, to the extent labeled, will be labeled with the same reference number augmented by a prime (′).

More specifically, the first pocket portion 52 defines base wall portion 56, back wall portion 58 and side wall portion 59. The portions of the first pocket portion define a pocket cavity 51 that is essentially formed into the upstanding wall. The second pocket portion 54 is substantially identical thereto.

The upper flange is positioned at the distal end of the upstanding wall and defines an axially outward configuration defining upper surface 60 and outer rim 62. In the configuration shown, the base 30, upstanding wall 32, upper flange 34 and lower hose attachment interface are integrally molded as a single unit. In other configurations, these structures may comprise multiple separate components that are adhered or otherwise joined together, while, it is preferred due to cost considerations that the structure is an integrally formed (i.e., injection or additive) member. It will be understood that at least a portion, and, preferably the entirety of the base comprises a transparent and/or translucent structure such that the test strips are visible through the upstanding wall (and/or the base and/or the upper flange).

With reference to FIGS. 2 through 4 the test strip 22 comprises any one of a number of different structures which can wick or otherwise, through capillary action pull fluid thereinto, and which are configured to include chemical additives which will provide a visual indication in the presence of a particular chemical, set of chemicals or other materials within the urine (or fluid stream). Some such test strips and test strip materials are disclosed in the incorporated '299 and '298 patents, which are set forth above, and incorporated herein by reference. Each test strip, such as test strip 22 includes a first portion 70 and a second portion 72. The portions are formed by bending the test strip so that first portion 70 and second portion 72 are disposed at an angle relative to each other, with such angle being substantially a right angle (+/−10%), preferably, while other oblique relative configurations are contemplated. The second portion 72 terminates with end 74. As will be explained below, the test strip 22 is positionable within the first pocket portion 52 of the housing 20.

With reference to FIGS. 4 and 6, he hose coupling 24 is shown as defining an elongated member 80 having a central bore 90, with the hose coupling 24 being attachable to the housing 20 to capture the test strips 22 therebetween, and providing a bore extending through the entirety of the connector assembly. More specifically, the hose coupling 24 includes central flange 82, depending wall 84 and hose connector 86. In the configuration shown, the hose coupling 24 comprises an integrally formed member (i.e., injection or additive) of a polymer material. In the configuration shown, the hose coupling comprises an opaque material, while it is contemplated that the hose coupling may comprise a transparent or translucent material. It is contemplated that the color of the hose coupling may be varied so as to amplify or otherwise clarify, improve or positively impact the ability to visually identify the necessary condition of the test strips to confirm the presence and/or absence of an infection.

The central flange 82 of the hose coupling 24 includes an upper surface 91 and a lower surface 92 opposite the upper surface 91. In the configuration shown, the central flange 82 substantially matches the configuration of the upper surface 60 of the upper flange.

The depending wall 84 extends in a first direction from the lower surface 92 of the central flange 82. The depending wall 84 includes proximal end 93, distal end 94, defining outer surface 95 and distal annular surface 96. In the configuration shown, the outer surface 95 of the depending wall 84 comprises a cylindrical configuration generally matching the circular configuration of the first portion 50 of the upstanding wall 32.

The hose connector 86 is shown as extending in a second direction from the upper surface 91 of the central flange 82. The hose connector 86 comprises outer surface 100 and bore 102. The outer surface 100 includes a plurality of step downs 104, which are a common configuration of an outer surface that is to sealingly engage the end of a catheter or other hose.

With reference to FIGS. 1 and 4, to assemble the extension assembly of the present disclosure, the user is first provided with the components of the connector assembly 12 and, also, the hose 14. With the components of the connector assembly, the housing 20 is first provided. Next, the test strips are formed into the desired configuration, which in the configuration shown, comprises a bent right angle between the first portion 70 and the second portion 72. The first of the test strips is then positioned within the first pocket portion cavity 51 so that the second portion 72 extends over the base wall portion 56 (i.e., the upper surface of the base 30) proximate opening 46 (wherein a portion of the test strip may overlay a portion of the opening 46 in some configurations). The second of the test strips is then positioned within the second pocket portion cavity 54 in the same manner. In the configuration shown, due to the shape of the test strips, the second portions may overlap slightly. In other configurations, the second portions may be shaped so that there is no such overlap.

Once the test strips are positioned, the hose coupling is attached to the housing. In greater detail, the depending wall 84 of the hose coupling 24 is inserted into the base cavity 49 of the housing 20. As the insertion continues, the lower surface 92 of the central flange 82 contacts the upper surface 60 of the outer flange 34 precluding further insertion. In such a fully inserted configuration, the distal annular surface 96 of the depending wall 84 preferably interfaces with the second portions of the test strips, and may provide a compressive force thereagainst, while covering a portion thereof in some configurations. As will be understood that outer surface 95 of the depending wall protects and isolates the remainder of the test strips from the stream of fluid extending through the central bore 90, while exposing the ends of the second portion of the test strips.

In the configuration shown, adhesive may be applied to the lower surface of the central flange prior to insertion. In other configurations, adhesive may be supplied into the well created by the upper flange 34 and the central flange of the hose coupling 24, and in greater detail, the well bounded by the outer rim 62 and the upper surface 60 of the upper flange 34 and the upper surface 91 of the central flange, along with a portion of the outer surface 100 of the hose connector 86.

Once secured and fully assembled, the connector assembly can be attached to hose 14. In some configurations, the connector assembly may be provided with a hose, or without a hose. To attach the two together, the end of the hose is extended over the outer surface 60 of the lower hose attachment interface 36. In some configurations, the coupling may be an interference fit, whereas in other configurations, an adhesive may be utilized to provide enhanced attachment between the hose and the connector assembly.

In use, as fluid (urine) passes through the bore of the connector assembly, preferably from a catheter or the like that is attached to the hose connector 86 of the hose coupling 24, a portion of the fluid that is passing contacts the second ends of the test strips. Due to the material of the test strips, the fluid wicks into the first and second portions of the test strips. As the test strips have certain chemical additives that are configured to change color in the presence of constituents in the urine that would be indicative of an infection, if an infection is present, the test strips will change in color providing a visual indication of infection.

While one embodiment of the present disclosure is useful for screening for urinary tract infection through detection of leukocytes and nitrites, the structural design is not limited to these diagnostic applications. The device may also be configured to incorporate other diagnostic test strips or assays capable of detecting additional biomarkers in urine, including but not limited to: glucose, ketones, protein, blood, bilirubin, urobilinogen, pH, and specific gravity. These markers are clinically relevant for monitoring conditions such as diabetes, kidney disease, liver dysfunction, and metabolic or hydration status. The adaptable nature of the device allows for future expansion of its diagnostic capabilities beyond infection screening, supporting broader clinical applications through urine-based biomarker detection.

Advantageously, as a majority of each of the test strips are isolated from the fluid flow, the chemical additives of the test strips will not tend to be washed away by the fluid stream, and, instead, only a small portion of fluid will reach the chemical additives of the test strips (while a substantial fluid flow may pass through the extension assembly itself).

Additionally, and also advantageously, the device shown herein is cost effective from a molding and assembly standpoint, thereby improving the ability to provide the same. Furthermore, the configuration of the housing and the hose coupling minimizes leakage of the connector assembly, while maximizing the operation of the test strips and the access to the test strips.

The foregoing description merely explains and illustrates the disclosure and the disclosure is not limited thereto except insofar as the appended claims are so limited, as those skilled in the art who have the disclosure before them will be able to make modifications without departing from the scope of the disclosure.

1. An extension assembly comprising:
a connector assembly;
a hose coupling.

2. (canceled)

3. (canceled)

4. (canceled)

5. (canceled)
